(19) United States
(12) Patent Application Publication
(10) Pub. No.: US 2020/0155048 A1
(43) Pub. Date: May 21, 2020

(54) BIOFLUID SENSING DEVICES WITH PH-BUFFERED EAB SENSORS

(71) Applicant: Eccrine Systems, Inc., Cincinnati, OH (US)

(72) Inventors: Jacob A Bertand, Montgomery, OH (US); Brian Hanley, Sacramento, CA (US); Mikel Larson, Cincinnati, OH (US); Gavi Begtrup, Cincinnati, OH (US)

(73) Assignee: Eccrine Systems, Inc., Cincinnati, OH (US)

(21) Appl. No.: 16/623,713

(22) PCT Filed: Jun. 20, 2018
(86) PCT No.: PCT/US18/38633

§ 371 Date: Dec. 17, 2019

Related U.S. Application Data
(60) Provisional application No. 62/522,762, filed on Jun. 21, 2017.
(60) Provisional application No. 62/634,220, filed on Feb. 23, 2018.

(51) Int. Cl. A61B 5/145; G01N 1/28; A61B 5/1477

(52) U.S. Cl. CPC A61B 5/14546; G01N 1/28; A61B 5/14517; A61B 5/1451; A61B 5/1477

(57) ABSTRACT

Devices and methods for tuning biofluid sample pH to enable more accurate analyte concentration measurements with pH-sensitive biosensors. In the embodiments, biofluid samples react with a polymer buffering material during transfer to a sensing element. The reaction with the buffering material causes protonation or deprotonation of the sample based upon 1) the pH of the sample, and 2) the selected quantity and pKa of the functional groups in the buffering material. Controlling the H+ content of a biofluid sample has beneficial effects on the accuracy of the biosensor by reducing or eliminating signal changes due to redox moiety variability, thereby isolating signal changes reflecting analyte concentration.

### CROSS-REFERENCE TO RELATED APPLICATIONS

The present application claims priority to PCT/US18/38633, filed Jun. 20, 2018, U.S. provisional application No. 62/522,762 filed on Jun. 21, 2017, and U.S. provisional application No. 62/634,220 filed on Feb. 23, 2018, the disclosures of which are hereby incorporated herein by reference in their entirety.

### BACKGROUND OF THE INVENTION

Despite the many ergonomic advantages of perspiration (sweat) compared to other biofluids (particularly in “wearable” devices), sweat remains an underutilized source of biomarker analytes compared to the established biofluids: blood, urine, and saliva. Upon closer comparison to other non-invasive biofluids, the advantages may even extend beyond ergonomics: sweat might provide superior analyte information. Sweat has many of the same analytes and analyte concentrations found in blood and interstitial fluid. Interstitial fluid has even more analytes nearer to blood concentrations than sweat does, especially for larger sized and more hydrophilic analytes (such as proteins).

A number of challenges, however, have historically kept sweat from occupying its place among the preferred clinical biofluids. These challenges include very low sample volumes (nL to μL), unknown concentration due to evaporation, filtration and dilution of large analytes, mixing of old and new sweat, and the potential for contamination from the skin surface. More recently, rapid progress in “wearable” sweat sampling and sensing devices has resolved several of the historical challenges. However, this recent progress has been limited to high concentration analytes (μM to mM) sampled at high sweat rates (>1 nL/min/gland) found in, for example athletic applications. Progress will be much more challenging as sweat biosensing moves towards detection of large, low concentration analytes (nM to pM and lower).

Additionally, many known sensor technologies for detecting larger molecules are ill-suited for use in wearable sweat sensing devices, which require sensors that permit continuous use on a wearer's skin. Therefore, sensor modalities that require complex microfluidic manipulation, the addition of reagents, the use of limited shelf-life components, such as antibodies, or sensors that are designed for a single use will not be sufficient for sweat sensing.

Electrochemical aptamer-based (“EAB”) biosensor technology, such as is disclosed in U.S. Pat. Nos. 7,803,542 and 8,003,374, presents a stable, reliable bioelectric sensor that is sensitive to target analytes in sweat, while also being capable of multiple analyte capture events during the sensor lifespan. As disclosed in PCT/US17/23399, incorporated by reference in its entirety herein, EAB sensors for use in continuous sweat sensing are configured to provide stable sensor responses with a life cycle extensive enough for multiple analyte binding and release cycles. Such sensors include a plurality of individual aptamer sensing elements, as depicted in FIG. 1A, which can repeatedly detect the presence of a molecular target by capturing and releasing target analytes as they establish equilibrium with the aptamer. The sensing element 110 includes an analyte capture complex 112 that includes a selected aptamer 140, and may include a linking section 142. The analyte capture complex 112 has a first end covalently bonded to a binding component. This binding component can include a sulfur molecule (thiol) 120, which is in turn covalently bonded to a gold electrode base 130. In other embodiments (not shown), the analyte capture complex may be bound to the electrode by means of an ethylenediaminetetraacetic acid (EDTA) strain, to improve adhesion in difficult sensing environments, such as sweat biofluid. The sensing element further includes a redox moiety 150 bound to a second end of the analyte capture complex 112. The redox moiety 150 may be covalently bonded to the aptamer 140, or bound to it by a linking section. In the absence of the target analyte, the aptamer 140 is in a first configuration, and the redox moiety 150 is in a first position relative to the electrode 130. When the sweat sensing device interrogates the sensing element using square wave voltammetry (SWV), the sensing element produces a first electrical signal.

With reference to FIG. 1B, the aptamer 140 is selected to specifically interact with a target analyte 160. When the aptamer captures a target analyte molecule, the aptamer undergoes a conformation change that partially disrupts the first configuration and forms a second configuration. The capture of the target analyte 160 accordingly moves the redox moiety 150 into a second position relative to the electrode 130. Now when the sweat sensing device interrogates the sensing element, the sensing element produces a second electrical signal that is distinguishable from the first electrical signal. After an interval of nanoseconds, milliseconds, seconds or longer, (the “recovery interval”), the aptamer releases the target analyte, and returns to the first configuration, which will produce the corresponding first electrical signal when the sensing element is interrogated.

Current state of the art EAB sensors use a methylene blue (MB) molecule as a redox moiety, because its behavior is well understood, it has a suitably low redox reaction potential, and it is stable during typical electrochemical processes. In testing media with very stable and narrow pH ranges, such as blood, aptamer sensing elements using MB as the redox moiety have very consistent performance through multiple signal-on/signal-off analyte capture cycles. One challenge with the use of EAB sensor technology for sweat sensing, however, is that electrical outputs from such sensors often have a strong dependence on pH. Sweat pH is not stable, and can vary as much as 300×, from about 4.5 to about 7. Because of the nature of its redox reaction, MB's performance is very sensitive to the variation in sweat sample pH. Methylene blue's redox potential depends both on its protonation state and, as depicted in FIG. 2, its reliance upon a proton (H+) transfer to perform the redox reaction. Therefore, MB is doubly sensitive to a sweat sample's H+ concentration, and the pH of a sample must be known to properly correlate a measured signal to a concentration of the target analyte.

One solution to mitigate the effect of pH variability on EAB sensor response would be to add a pH sensor and use the readings from the sensor to correct for pH-induced errors in the response. For example, integration of a pH sensor in a sweat sensing device is disclosed in PCT/US15/40113, which is hereby incorporated herein in its entirety. For many applications, however, adding sensors to correct for pH may prove inferior to buffering the biofluid sample to reduce the effects of pH variability on the EAB sensors. Devices and methods for buffering a sweat sample for pH are disclosed in PCT/US16/58357, which employ a membrane with minimal pore sizes to prevent the buffer molecules, such as phosphorus and sulfate, from traversing the membrane and contaminating the sweat sample. However, such use of a buffer membrane with minimal pore size prevents or substantially slows adequate, real time proton transfer between the buffer solution and the biofluid sample and, thus, negatively impacts the sampling rate.

Accordingly, it is desirable to have simple, yet robust devices and methods to reduce the output variability due to pH for one or more redox mediated sensors in a wearable biofluid sensing device. It is particularly desirable to have devices and methods for tuning the pH of a biofluid sample rapidly, with minimal impedance of sample flow rate, prior to reaction of the sample with one or more aptamer sensing elements. Rapid, real time tuning of biofluid sample pH, as the sample is collected, improves the accuracy and stability of the EAB sensor across multiple analyte capture cycles.

### SUMMARY OF THE INVENTION

Devices and methods are described herein for tuning biofluid sample pH to enable more accurate analyte concentration measurements with pH-sensitive sensors. In the disclosed embodiments, biofluid samples react with a polymer buffering material during transfer to a sensing element. The reaction with the buffering material causes protonation or deprotonation of the sample based upon 1) the pH of the sample, and 2) the selected quantity and pKa of the functional groups in the buffering material. Controlling the H+ content of a biofluid sample has beneficial effects on the accuracy of the biofluid sensor by reducing or eliminating signal changes due to redox moiety variability, thereby isolating signal changes reflecting analyte concentration.

### BRIEF DESCRIPTION OF THE DRAWINGS

The objects and advantages of the disclosed invention will be further appreciated in light of the following detailed descriptions and drawings in which:

FIGS. 1A and 1B are representations of a previously-disclosed EAB sensing element;

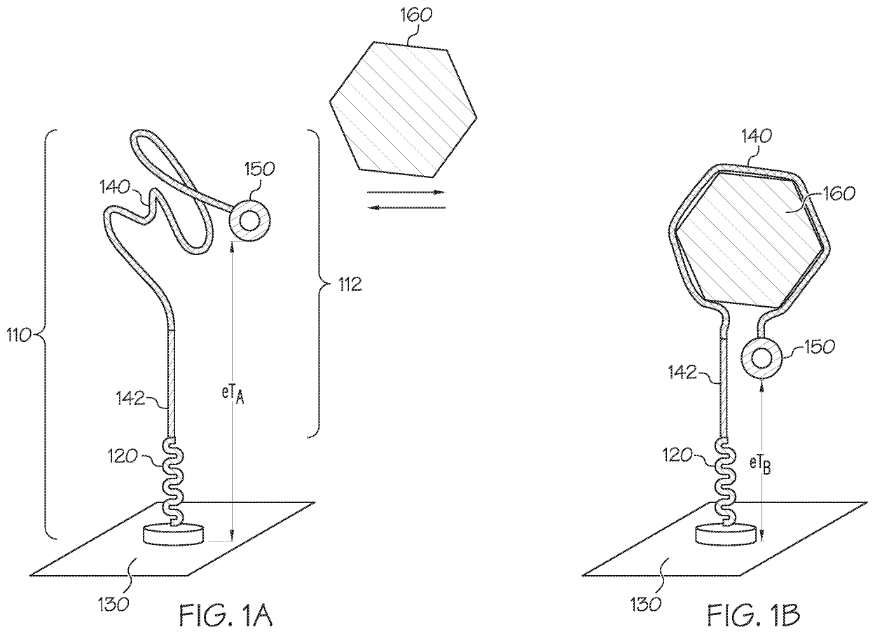

FIG. 2 depicts variations in a methylene blue (MB) molecule at differing pH ranges;

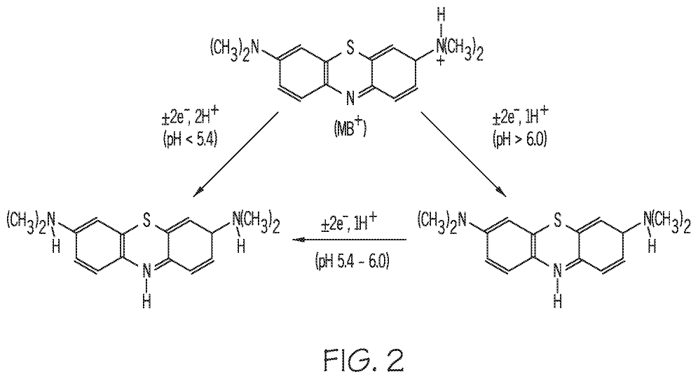

FIG. 3 is a schematic depiction of a wearable sensing device configured to accomplish chemical buffering of biofluid samples;

FIG. 4 depicts a representative zwitterion functional group applicable in the disclosed device;

FIG. 5 is a schematic depiction of a second embodiment of a wearable sensing device configured to buffer biofluid samples;

FIG. 6 is a schematic depiction of a third embodiment of a wearable sensing device configured to buffer biofluid samples;

FIG. 7A is a schematic depiction of a fourth embodiment of a wearable sensing device configured to buffer biofluid samples;

FIG. 7B is a cross-sectional view of a microfluidic channel taken along line 7B-7B in FIG. 7A;

FIG. 8A is a schematic depiction of a fifth embodiment of a wearable sensing device configured to buffer biofluid samples;

FIG. 8B is a cross-sectional view of a microfluidic channel taken along line 8B-8B in FIG. 8A;

FIG. 9A is a schematic depiction of a sixth embodiment of a wearable sensing device configured to buffer biofluid samples; and

FIG. 9B is a cross-sectional view of a microfluidic channel taken along line 9B-9B in FIG. 9A.

### DEFINITIONS

“Continuous monitoring” means the capability of a device to provide at least one measurement of biofluid determined by a continuous or multiple collection and sensing of that measurement or to provide a plurality of measurements of biofluid over time.

As used herein, “interstitial fluid” or “tissue fluid” is a solution that bathes and surrounds tissue cells. The interstitial fluid is found in the interstices between cells. Embodiments of the disclosed invention measure analytes from interstitial fluid found in the skin and, particularly, interstitial fluid found in the dermis. In some cases where interstitial fluid is emerging from sweat ducts, the interstitial fluid contains some sweat as well, or alternately, sweat may contain some interstitial fluid.

As used herein, “biofluid” may mean any human biofluid, including, without limitation, sweat, interstitial fluid, blood, plasma, serum, tears, and saliva. For sweat sensing applications as generally discussed herein, biofluid has a narrower meaning, namely, a fluid that is comprised mainly of interstitial fluid or sweat as it emerges from the skin.

As used herein, “chronological assurance” means the sampling rate or sampling interval that assures measurement(s) of analytes in a biofluid are made at the rate where measurements can be made of new biofluid analytes emerging from the body. Chronological assurance may also include a determination of the effect of sensor function, potential contamination with previously generated analytes, other fluids, or other measurement contamination sources. Chronological assurance may have an offset for time delays in the body (e.g., a well-known 5 to 30-minute lag time between analytes in blood emerging in interstitial fluid), but the resulting sampling interval (defined below) is independent of lag time, and furthermore, this lag time is inside the body, and therefore, for chronological assurance as defined above and interpreted herein, this lag time does not apply.

As used herein, “biofluid sampling rate” or “sampling rate” is the effective rate at which new biofluid, originating from pre-existing pathways, reaches a sensor that measures a property of the fluid or its solutes. Sampling rate is the rate at which new biofluid is refreshed at the one or more sensors and therefore old biofluid is removed as new fluid arrives. In one embodiment, this can be estimated based on volume, flow-rate, and time calculations, although it is recognized that some biofluid or solute mixing can occur. Sampling rate directly determines or is a contributing factor in determining the chronological assurance. Times and rates are inversely proportional (rates having at least partial units of 1/seconds), therefore a short or small time required to refill sample volume can also be said to have a fast or high sampling rate. The inverse of sampling rate (1/s) could also be interpreted as a “sampling interval(s)”. Sampling rates or intervals are not necessarily regular, discrete, periodic, discontinuous, or subject to other limitations. Like chronological assurance, sampling rate may also include a determination of the effect of potential contamination with previously generated biofluid, previously generated solutes (analytes), other fluid, or other measurement contamination sources for the measurement(s). Sampling rate can also be in part determined from solute generation, transport, advective transport of fluid, diffusion transport of solutes, or other factors that will impact the rate at which new sample will reach a sensor and/or is altered by older sample or solutes or other contamination sources.

As used herein, “sample generation rate” is the rate at which biofluid is generated by flow through pre-existing pathways. Sample generation rate is typically measured by the flow rate from each pre-existing pathway in nL/min/pathway. In some cases, to obtain total sample flow rate, the sample generation rate is multiplied by the number of pathways from which the sample is being sampled. Similarly, as used herein, “analyte generation rate” is the rate at which solutes move from the body or other sources toward the sensors.

As used herein, “measured” can imply an exact or precise quantitative measurement and can include broader meanings such as, for example, measuring a relative amount of change of something. Measured can also imply a binary measurement, such as ‘yes’ or ‘no’ type qualitative measurements.

As used herein, “sample volume” is the fluidic volume in a space that can be defined multiple ways. Sample volume may be the volume that exists between a sensor and the point of generation of biofluid sample. Sample volume can include the volume that can be occupied by sample fluid between: the sampling site on the skin and a sensor on the skin where the sensor has no intervening layers, materials, or components between it and the skin; or the sampling site on the skin and a sensor on the skin where there are one or more layers, materials, or components between the sensor and the sampling site on the skin.

“Analyte” means a substance, molecule, ion, or other material that is measured by a biofluid sensing device.

As used herein, the term “analyte-specific sensor” is a sensor specific to an analyte and performs specific chemical recognition of the analytes presence or concentration (e.g., ion-selective electrodes, enzymatic sensors, electrochemical aptamer-based sensors, etc.). For example, sensors that sense impedance or conductance of a fluid, such as sweat, are excluded from the definition of “analyte-specific sensor” because sensing impedance or conductance merges measurements of all ions in biofluid (i.e., the sensor is not chemically selective; it provides an indirect measurement). Sensors could also be optical, mechanical, or use other physical/chemical methods which are specific to a single analyte. Further, multiple sensors can each be specific to one of multiple analytes.

“EAB sensor” means an electrochemical aptamer-based biosensor that is configured with multiple aptamer sensing elements that, in the presence of a target analyte in a biofluid sample, produce a signal indicating analyte capture, and which signal can be added to the signals of other such sensing elements, so that a signal threshold may be reached that indicates the presence or concentration of the target analyte.

“Aptamer” means an aptamer, a DNA aptamer, or other suitable molecules or complexes, such as proteins, polymers, molecularly imprinted polymers, polypeptides, and glycans, that experience a conformation change in the presence of a target analyte and are capable of being used in an analyte-specific sensor.

“Sensitivity” means the change in output of the sensor per unit change in the parameter being measured. The change may be constant over the range of the sensor (linear), or it may vary (nonlinear).

“Recovery interval” means the time required for an aptamer sensing element to release a target analyte back into solution and return to its signal-off configuration.

“Signal threshold” means the combined strength of signal-on indications produced by a plurality of aptamer sensing elements that indicates the presence of a target analyte.

### DETAILED DESCRIPTION OF THE INVENTION

Referring now to the drawing figures, in which like numerals indicate like elements throughout the views, several exemplary embodiments of a biofluid sensing device and method will be described. Embodiments described herein apply to any type of biofluid sensing device that measures at least one analyte in sweat, interstitial fluid, or other biofluid. The disclosed embodiments may be applied to sensing devices which measure samples at chronologically assured sampling rates or intervals. Further, the embodiments described herein may take on many forms including patches, bands, straps, portions of clothing, wearables, or any other suitable mechanism that reliably brings sampling and sensing technology into intimate proximity with one or more biofluid samples as the sample is transported to the skin surface. While some embodiments utilize adhesives to hold a sensing device near the skin, devices could also be held by other mechanisms that hold the device secure against the skin, such as a strap or embedding in a helmet. Certain embodiments show sensors as simple individual elements. It is understood that many sensors require two or more electrodes, reference electrodes, or additional supporting technology or features which are not captured in the description herein. Sensors are preferably electrical in nature, but may also include optical, chemical, mechanical, or other known biosensing mechanisms. Sensors can be in duplicate, triplicate, or more, to provide improved data and reading accuracy. Certain embodiments of the disclosed invention show sub-components of what would be sensing devices with more conventional sub-components (such as a battery) needed for use of the device in various applications. For purposes of brevity and of greater focus on inventive aspects, such subcomponents are not explicitly shown or described herein.

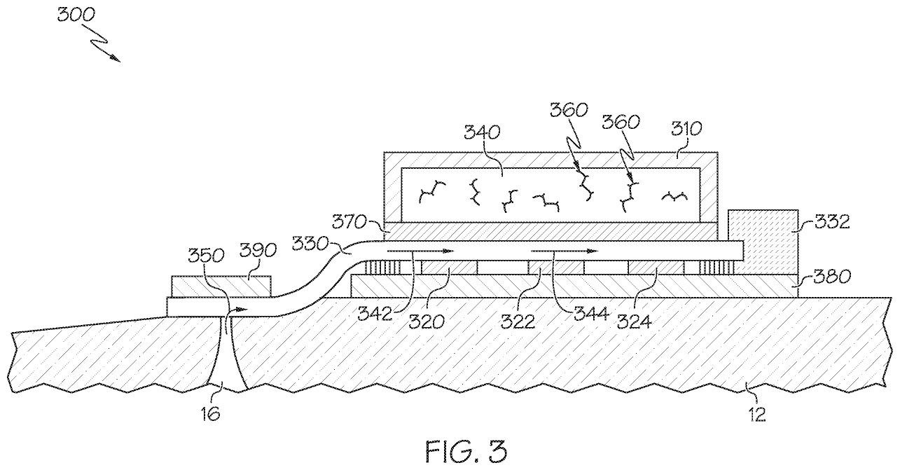

Referring now to FIG. 3, which illustrates a first embodiment of a biofluid sensing device 300 shown on a section of skin 12. The device 300 includes at least one analyte-specific sensor (three sensors 320, 322, 324 are shown in the illustrated embodiment). The device further includes a polymer substrate 380 made of PET, or other suitable material, on the skin surface 12. A microfluidic channel 330 contacts the skin surface, or is in fluid communication with the skin surface through a sweat collector, for accruing one or more sweat and/or other biofluid samples, indicated by arrow 350, as the sample emerges from a gland 16. The biofluid sample is conveyed through the channel 330, as indicated by the arrows 342, 344, past sensors 320, 322, 324, and onto a sample pump 332. The sample can be conveyed through channel 330 by any suitable mechanism for transport, including osmosis or wicking pressures. The microfluidic channel 330 may comprise a closed channel, an open channel, a tubular passage which may be partially or fully enclosed, a paper or textile wick, or other similar apparatus or materials. Similarly, the sample pump 332 may comprise a paper or textile wick, or other materials. Some embodiments may include a sweat stimulation component 390, comprising iontophoresis electrodes and a sweat stimulation chemical, to facilitate sweat sweating at desired times, or from sedentary individuals.

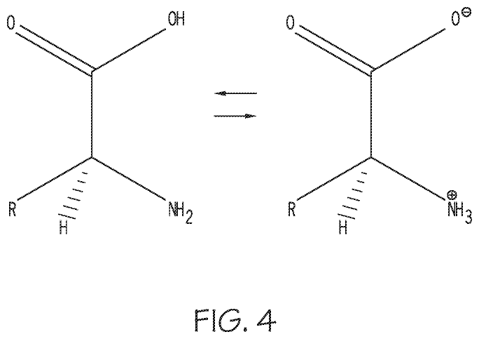

The embodiments described herein include a buffering material for tuning the pH of a biofluid sample on exposure of the sample to the buffering material. The buffering material may comprise any individual or polyfunctional polymer chain having one or more functional groups that regularly accept or donate a hydrogen ion in response to the pH of a substance in fluidic contact with the material. The functional groups can be ionic or non-charged depending upon the pH of the biofluid sample and pKa of the selected functional groups. The buffering material will tune the sample's H+ concentration to an operative or preferred pH for the EAB sensor by protonating or deprotonating the sample, depending on whether the sample is a higher or lower pH than the buffering material. The buffering material may stabilize biofluid samples for two or more different pH levels through the use of different functional groups on the polymer chain, or by using copolymers. The functional groups may include polyprotic moieties, thereby allowing for two or more buffering regions to exist on the titration curve of the material. Samples having different pH levels can be buffered to the pH of the sensor by altering the number of protons dissociated from the sample. The selected polymer(s) will vary, depending upon the application, by altering the number of functional groups and/or the pKa values of the functional groups on the polymer chain or co-polymers. The selection of a suitable buffering material involves a balance between the desired signal from the EAB sensor, the density of the polymers, the flow rate of the biofluid sample, and the desired time resolution for the sensor. FIG. 4 depicts one set of exemplary polymer moieties suitable for use as a buffering material. The zwitterion depicted in FIG. 4 is a high molecular weight polymer chain having two or more selected functional groups, including both acidic and basic groups. The exemplary zwitterion is configured with functional groups that include an amino acid having a carboxylic acid fragment and an amine fragment. In the example shown in FIG. 4, the buffering material has a pH of approximately 6.8, and would be suitable for buffering samples for applications using aptamer sensing elements that have an operative pH of 6.8.

In the device depicted in FIG. 3, a buffering material 340, comprising one or more polymers 360 in an aqueous solution or gel, is retained within a reservoir 310. A semi-permeable membrane 370 extends across and seals an open side of the reservoir 310. The membrane 370 has a first side in fluidic communication with the buffering material 340, and a second side in fluidic communication with the microfluidic channel 330. The buffering material 340 interacts with the biofluid sample in channel 330 through membrane 370. To facilitate tuning of the biofluid sample, membrane 370 is selected to have a porosity that allows for proton (H+) exchange between the buffering material 340 and the biofluid sample, but which is otherwise impermeable to the buffer molecules or analyte molecules. The buffering material and membrane are selected to optimize the H+ exchange with a minimum membrane surface area between the fluid channel and buffering material. This relationship is governed according to the equation:

$$\frac{d ( H + )}{d t} ∝ \frac{Area ( Pore size )}{Vf} ,$$

which indicates that the rate of exchange between the buffering material and sample will depend directly upon the surface area and pore size of the membrane, and inversely on the velocity of the fluid sample past the membrane. When the biofluid sample has a pH above the pH of the buffering material 340, hydrogen ions will be transferred from molecules of the buffering polymers 360 to molecules in the biofluid sample. This H+ transfer adjusts the sample pH to the buffer pH, giving the buffering material 340 a net negative or net neutral charge. Likewise, when the biofluid sample has a pH that is lower than the pH of the buffering material 340, hydrogen ions will be removed from the sample molecules and transferred to the buffer molecules, giving the buffering material a net positive or net neutral charge. The buffering material 340 will have a much larger volume relative to the biofluid sample volume, providing sufficient capacity to stabilize biofluid samples through numerous sampling intervals, without losing the proton transfer capability of the buffer. In the embodiment depicted in FIG. 3, the free-floating condition of the polymer within a solution allows a simplified manufacturing process, in which the buffering material can be prepared without the need to attach the polymer molecules to a casing or substrate.

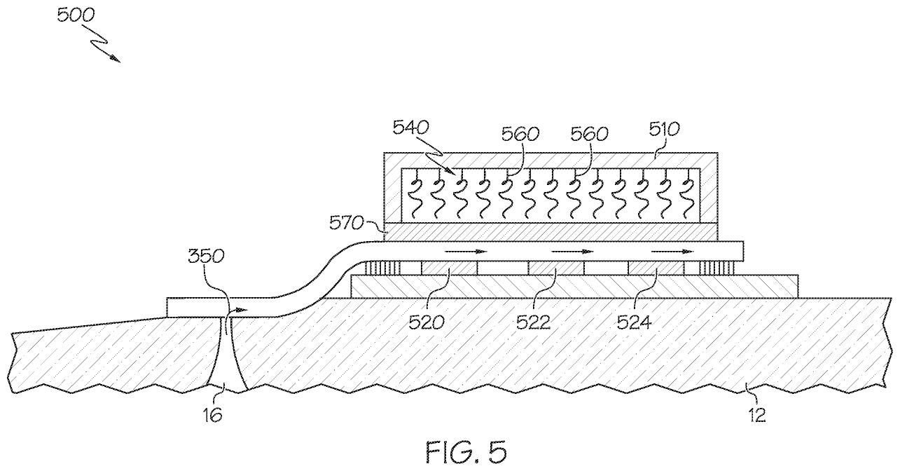

In a second embodiment, depicted in FIG. 5, a sensing device 500 includes a buffering material 540 in an immobilized condition. In this embodiment, the buffering material includes one or more selected polymers 560 chemically fixed on a surface within a reservoir 510. The polymer molecules 560 may be affixed by covalent bond, or other suitable method known in the art. Bonding the polymer chains to an inner surface of the reservoir 510 allows greater flexibility to increase the pore size in a membrane 570. The fixed state of the polymer molecules 560 within the reservoir 510 allows the sample to react with the polymer while preventing molecules from moving through the larger-sized membrane pores to contaminate the sample. For certain applications, the larger membrane pore size also allows for a relatively quicker ion exchange between the sample and buffer polymer.

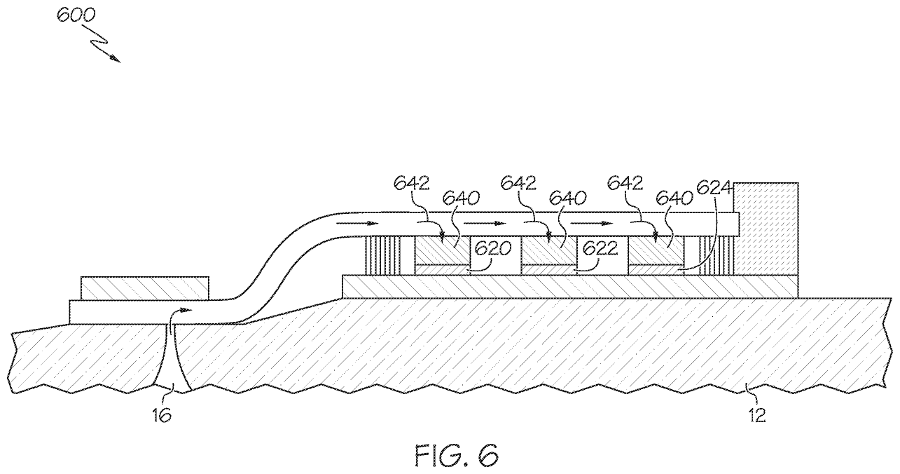

In a third embodiment, depicted in FIG. 6, a sensing device 600 includes buffering material 640 localized to individual EAB sensors (three sensors 620, 622, 624 are shown in the illustrated embodiment), in order to vary the pH environment of the individual sensors. The one or more polymers in buffering material 640 are selected to tune the sample pH to a preferred, operative pH for the aptamer sensing elements of the individual sensor. A polymer is solvent cast onto each individual sensor 620, 622, 624 to surround the aptamer sensing elements in buffering material. As a biofluid sample moves through channel 330, a portion of the sample will diffuse through the buffering material 640, as indicated by arrows 642, before interacting with the individual sensors 620, 622, 624. As the sample diffuses through the buffering material 640, the sample is protonated or deprotonated, as described above, to achieve substantial equivalence of pH between the sample and the buffering material. The polymer density of the buffering material 640 is selected to achieve buffering of the sample pH prior to the sample reacting with the sensor. Different polymers and densities can be selected for the buffering material for each of the separate sensors 620, 622, 624. Localizing the buffering material 640 to individual sensors 620, 622, 624 enables the sample pH to be tuned to a different equilibrium pH for each sensor. Localizing the buffering material 640 to the individual sensors also eliminates the need for a buffering reservoir and membrane as in the previous embodiments.

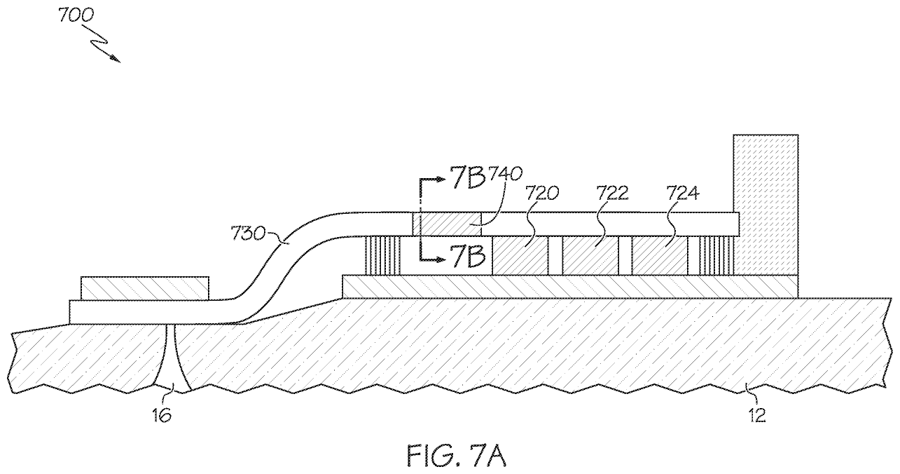

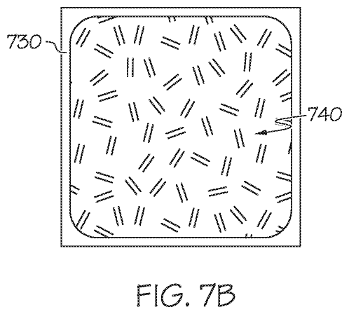

In another exemplary embodiment, depicted in FIGS. 7A and 7B, a sensing device 700 includes a buffering material 740 within a microfluidic channel 730 for tuning a biofluid sample's pH as the sample flows through the channel. The buffering material 740 is positioned within the channel 730 so as to fluidly contact the sample prior to the sample reaching the individual sensors 320, 322, 324. The buffering material 740, which comprises a polymer hydrogel or similar material, spans a cross-section of the channel to provide a surface area for interaction with the sample without impeding sample flow. Biofluid samples are driven through the buffering material 740 in the channel 730 by the positive pressure of the biofluid exiting a gland 16. The length, density, and polymer composition of the buffering material plug 740 are selected to adjust the sample pH to a desired pH level, prior to reacting with the sensing elements, while maintaining an adequate sampling rate for chronological assurance.

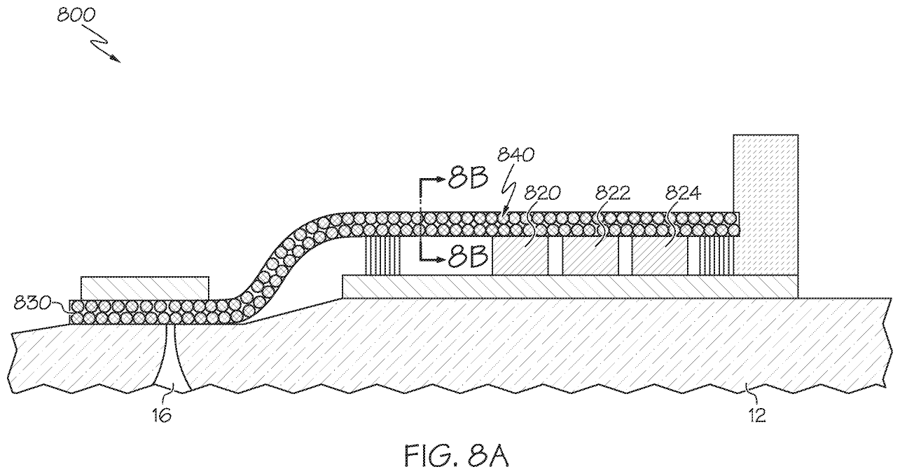

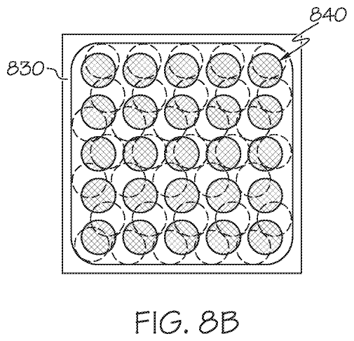

Alternatively, as shown in FIGS. 8A and 8B, a biofluid sensing device 800 of the disclosed invention includes a microfluidic channel 830 configured as a packed column having buffering polymers that are affixed on a substrate. The substrate can be in the form of a plurality of spherules 840 packed within the microfluidic channel 830 to react with the biofluid sample as the sample moves through the channel. The polymer coated substrate may also be any other suitable arrangement providing high surface area and minimal flow resistance, such as techniques used in low pressure liquid chromatography. The spherules or other substrate are coated with a polymer selected to adjust the biofluid sample to the desired pH for the one or more sensors 320, 322, 324. The polymers are selected to accomplish pH tuning of the sample at the sample flow rate, prior to the sample reaching a sensor. As a biofluid sample flows through the channel 830, the substrates 840 increase the surface contact between the sample and the buffering polymer, facilitating rapid proton exchange. Configuring the buffering material as a packed column may prove superior for certain applications, by allowing the device to buffer a larger sample more rapidly within the limited space of the microfluidic channel.

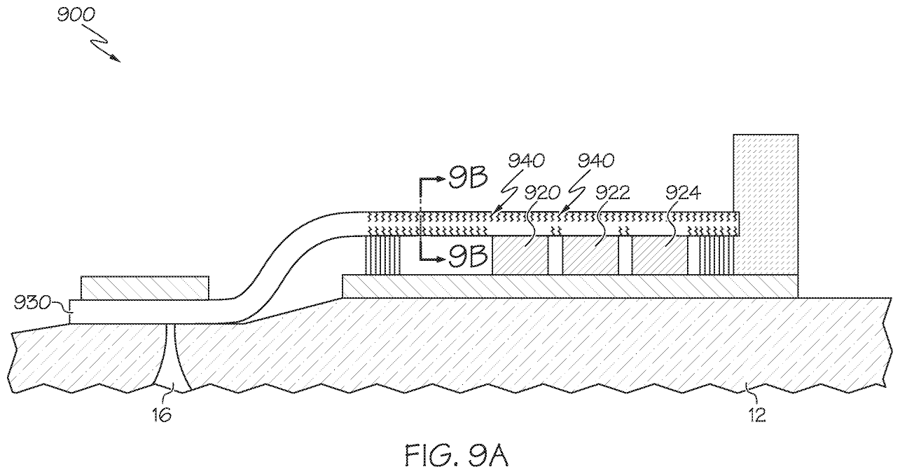

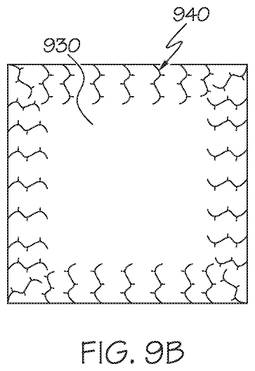

In another exemplary embodiment, depicted in FIGS. 9A and 9B, a sensing device 900 includes a buffering material 940 chemically affixed to an inner surface of a fluid channel 930 to directly contact a biofluid sample as the sample is conveyed through the channel. The buffering material 940 may be covalently bonded or otherwise chemically attached within the channel to substantially coat one or more inner surfaces of the channel. The buffering material preferably extends for substantially the length of the fluid channel 930, and coats as much of the inner channel surface as possible in order to maximize the fluidic contact between the buffering material and the sample. Openings may be provided through the buffering material 940 to allow the sample to flow to the sensors 320, 322, 324. As a biofluid sample flows through the channel 930 the polymer coating 940 reacts with molecules in the sample, transferring H+ from the substance having higher proton concentration to the substance with lower proton concentration. This H+ transfer between the polymer coating and biofluid sample buffers the sample pH in real time as the sample flows through the microfluidic channel and across the sensors 320, 322, 324.

In an additional, timed buffering embodiment, a pH sensor (not shown) at an inlet to a fluid channel measures the pH of an incoming biofluid sample. The sample pH measurement dictates a time interval for the sample to flow through the channel prior to the device reading a signal from the sensors 320, 322, 324. The greater the difference between the sample pH and the sensor pH, the greater the time interval prior to obtaining a sensor reading. Increasing the time interval provides additional time for buffering the sample within the fluid channel to achieve the desired sample pH prior to reading the sensor signal.

In each of the described embodiments, one or more polymers are selected to provide a rapid exchange of protons with a biofluid sample, in real time, as the sample is conveyed to a sensor. The devices disclosed herein improve upon previous pH buffers for EAB sensors by increasing the fluid contact between the buffer and the sample. Using a larger-sized polymer as a buffer, rather than phosphate or sulfate as used in prior known buffers, allows for larger pore size in a buffering membrane without molecules passing through the membrane and contaminating the sample. Additionally, configuring the buffer as one or more polymer chains, and affixing the polymer chains within a fluid channel or on a sensor, allows for device configurations that eliminate the buffering reservoir and membrane, while providing rapid H+ exchange between the buffer and biofluid sample to maintain the sampling rate. Eliminating the buffering reservoir and membrane reduces the bulk and complexity of the devices.

While several embodiments have been described herein, it should be apparent that various modifications, alterations, and adaptations to those embodiments may occur to persons skilled in the art with attainment of at least some of the advantages. The disclosed embodiments are therefore intended to include all such modifications, alterations, and adaptations without departing from the scope of the embodiments as set forth herein.

1. A biofluid sensing device comprising:
a sensor having a plurality of sensing elements, the sensor configured to measure a concentration of an analyte in a biofluid sample;
an electrode for producing one or more electrical signals, the electrical signals varying in response to a measurement of the analyte by the plurality of sensing elements;
a channel for conveying the biofluid sample to the sensor; and
a buffering material in fluidic contact with the biofluid sample, wherein the buffering material is configured to adjust a potential of hydrogen (pH) value of the sample to an operative sensor pH.

2. The device of claim 1, wherein the buffering material comprises at least one polymer having functional groups selected to adjust the pH value to the operative sensor pH.

3. The device of claim 2, wherein the buffering material comprises a polymer chain having one or more functional groups.

4. The device of claim 2, wherein the buffering material comprises two or more polymers having different functional groups.

5. The device of claim 2, wherein the buffering material further comprises a zwitterion.

6. The device of claim 1, further comprising a reservoir containing the buffering material.

7. The device of claim 6, further comprising a selectively permeable membrane in fluidic communication with the buffering material and the channel.

8. The device of claim 7, wherein the buffering material comprises at least one polymer in solution within the reservoir.

9. The device of claim 8, wherein the membrane has a pore size that allows hydrogen ions to pass through the membrane while retaining the buffering material in the reservoir.

10. The device of claim 1, wherein the buffering material is localized to a sensor environment.

11. The device of claim 1, wherein the buffering material adjusts the pH value as the biofluid sample moves through the channel.

12. The device of claim 10, wherein the buffering material surrounds a plurality of sensing elements to adjust the pH value prior to the biofluid sample reacting with the sensor.

13. The device of claim 1, wherein the buffering material comprises a plurality of polymer chains chemically attached to a surface of the device.

14. The device of claim 13, wherein the surface is one of within a reservoir, or inside the channel

15. (canceled)

16. The device of claim 1, wherein the buffering material contacts the biofluid sample in the channel to adjust the pH value prior to contact with between the biofluid sample and the sensor.

17. The device of claim 1, wherein the buffering material comprises one of: a packed column within the channel, or a hydrogel within the channel

18. (canceled)

19. A method of adjusting the potential of hydrogen (pH) of a biofluid sample in a sensing device, the method comprising:
collecting a biofluid sample containing at least one analyte;
conveying the biofluid sample through a channel to a sensor configured to measure a characteristic of the at least one analyte;
exposing the biofluid sample to a buffering material prior to contact with the sensor; and
using the buffering material to adjust a pH value of the biofluid sample to an operative pH.

20. The method of claim 19, wherein the buffering material comprises a polymer having a plurality of functional groups selected to adjust the pH value to the operative pH.

21. The method of claim 20, wherein the exposing step further comprises exposing the biofluid sample to the buffering material through a membrane.

22. The method of claim 20, wherein the polymer is a zwitterion.

23. The method of claim 20, further comprising attaching the polymer to a surface of the device.

24. The method of claim 23, further comprising attaching the polymer within the channel.

25. The method of claim 20, wherein the exposing step further comprises exposing the biofluid sample to the buffering material as the biofluid sample is conveyed through the channel.

26. The method of claim 25, wherein the exposing step further comprises configuring the buffering material on a surface of a substrate within the channel.

27. The method of claim 20, wherein the exposing step further comprises exposing the biofluid sample to the buffering material as the sample diffuses from the channel to the sensor.
